(19) United States
(12) Patent Application Publication
(10) Pub. No.: US 2024/0383826 A1
(43) Pub. Date: Nov. 21, 2024

(54) CONVERSION OF CHLOROFLUOROROPANES AND CHLOROFLUROPROPENES TO MORE DESIRABLE FLUOROPROPANES AND FLUOROROPENES

(71) Applicant: THE CHEMOURS COMPANY FC, LLC, Wilmington, DE (US)

(72) Inventor: XUEHUI SUN, Kennett Square, PA (US)

(73) Assignee: THE CHEMOURS COMPANY FC, LLC, Wilmington, DE (US)

(21) Appl. No.: 18/786,089

(22) Filed: Jul. 26, 2024

Related U.S. Application Data
(63) Continuation of application No. 17/722,504, filed on Apr. 18, 2022, now Pat. No. 12,060,309.
Application No. 17/722,504 is a continuation of application No. 17/025,109, filed on Sep. 18, 2020, now Pat. No. 11,332,424.
Application No. 17/025,109 is a continuation of application No. 16/775,308, filed on Jan. 29, 2020, now Pat. No. 10,815,173.
Application No. 16/775,308 is a continuation of application No. 16/435,615, filed on Jun. 10, 2019, now Pat. No. 10,584,082.
Application No. 16/435,615 is a continuation of application No. 15/786,199, filed on Oct. 17, 2017, now Pat. No. 10,351,495.
Application No. 15/786,199 is a continuation of application No. 15/466,148, filed on Mar. 22, 2017, now Pat. No. 9,822,047.
Application No. 15/466,148 is a continuation of application No. 14/680,092, filed on Apr. 7, 2015, now Pat. No. 9,637,429.
(60) Provisional application No. 61/980,157, filed on Apr. 16, 2014.

(51) Int. Cl. C07C 17/354; C07C 17/23; C07C 17/25; C07C 19/08; C07C 19/10; C07C 21/18

(52) U.S. Cl. CPC C07C 17/354; C07C 17/23; C07C 17/25; C07C 19/08; C07C 19/10; C07C 21/18

(57) ABSTRACT

A process is provided comprising contacting and reacting the compound CF₃CF₂CHXCl, wherein X is H or Cl, or the compound CF₃CF═CXCl, wherein X is H or Cl, with hydrogen in the presence of a catalyst consisting essentially of Cu, Ru, Cu—Pd, Ni—Cu, and Ni—Pd, to obtain as a result thereof reaction product comprising hydrofluoropropenes or intermediates convertible to said hydrofluoropropenes, notably CF₃CF═CH₂ and CF₃CH═CHF.

### FIELD OF THE INVENTION

The present invention relates to chemical processes for converting chlorofluoropropanes (HCFC) and chlorofluoropropenes (CFO) to more desirable fluoropropanes and fluoropropenes, especially propenes that are hydrofluoroolefins (HFO), i.e., free of Cl, and to intermediates from which these HFOs can be obtained.

### BACKGROUND OF THE INVENTION

U.S. Pat. No. 8,399,722 discloses the hydrogenation of at least one of 1,1-dichloro-2,3,3,3-tetrafluoropropene (CF₃CF═Cl₂, 1214ya) and 1-chloro-2,3,3,3-tetrafluoropropene (CF₃CF═CHCl, 1224yd) in the presence of catalyst composed of Pd supported on a carbon carrier to obtain 2,3,3,3,-tetrafluoropropene (CF₃CF═CH₂, 1234yf), which is free of chlorine and which has promise at least as a refrigerant exhibiting both low ozone depletion potential and low global warming potential. Example 2 discloses the conversion rate 1214ya to 1234yf to be 75%, which can also be considered the selectivity of the process. In Example 3 the selectivity dropped to 69%.

Processes are desired to obtain better results in the production of HFO-1234yf, and/or to obtain HFO-1234ze (CF₃CH═CHF), 1,3,3,3-tetrafluoropropene), which is also free of chlorine and has refrigerant application, and/or which opens up new routes for obtaining desirable HFOs such as HFO-1234yf or HFO-1234ze.

### SUMMARY OF THE INVENTION

The present invention provides the process comprising contacting and reacting the compound CF₃CF₂CHXCl, wherein X is H or Cl, or the compound CF₃CF═CXCl, wherein X is H or Cl, with hydrogen in the presence of a catalyst consisting essentially Cu, Ru, Cu—Pd, Ni—Cu, and Ni—Pd, to obtain as a result thereof reaction product comprising hydrofluoropropenes or intermediates convertible to said hydrofluoropropenes. When in the compound CF₃CF₂CHXCl, X is H, then the compound is HCFC-235cb, and when Cl, the compound is HCFC-225ca. When in the compound CF₃CF═CXCl, X is H, the compound is HCFO-1224yd, and when X is Cl, the compound is CFO-1214ya. These are the reactant compounds in this Hydrogenation Process.

Another embodiment of the present invention is the Intermediates Process, i.e., the process for converting the intermediates obtained in the Hydrogenation Process to the desired hydrofluoropropenes. The Intermediates Process comprises such reactions as hydrogenation, dehydrochlorination, and dehydrofluorination. The catalysts used in the Hydrogenation Process are preferably those used in the hydrogenation reactions in the Intermediates Process.

The desired hydrofluoropropenes include HFO-1234yf (CF₃CF═CH₂), HFO-1234ze (CF₃CH═CHF), and HFO-1225zc (CF₃CH═CF₂).

Reaction pathways (reactant/reaction product) included in the Hydrogenation Process and Intermediates Process are as follows:

When the reactant compound is HCFC-225ca, the reaction product comprises HFO-1234yf (CF₂CF═CH₂). The reaction product may also comprise at least one of HCFO-1224yd, and HCFC-235cb (CF₃CF₂CH₂Cl) which are intermediates through which HFO-1234yf can be obtained. HCFO-1224yd can be converted directly to HFO-1234yf. HCFC-235cb can be converted indirectly to HFO-1234yf by first being converted to HCFO-1224yd, which is then converted to HFO-1234yf. The HCFC-235cb can also be converted directly to HFO-1234yf, i.e., the reaction product of hydrogenating HCFC-235cb comprises HFO-1234yf. This direct conversion can result when the HCFC-235cb is the reactant compound CF₃CF₂CHXCl, wherein X is H.

Typically, the reactant compound HCFC-225ca will be mixed with HCFC-225aa (CF₃CCl₂CHF₂), whereby the HCFC-225aa will accompany the HCFC-225ca in the contacting and reacting step. The resultant hydrogenation of the HCFC-225aa forms the reaction product comprising at least one of HCFO-1224xe (CF₃CCl═CHF), HCFC-235da (CF₃CHClCHF₂), and HCFC-245fa (CF₃CH₂CF₂H), which are intermediates in the formation of HFO-1234ze (CF₃CH═CHF). The hydrogenation of HCFC-225aa can also form HFO-1234ze directly.

When the intermediate is HCFO-1234xe, the reaction pathway to HFO-1234ze is to first form HCFC-23db (CF₃CHClHCH₂F), which can be converted to HFO-1234ze or 1234ze can be formed directly from 1234xe. When the intermediate is HCFC-235da, this can be converted to HFO-1234ze. When the intermediate is HCFC-245fa, it can be converted to HFO-1234ze directly.

When the reactant compound is either CFO-1214ya or 1224yd or a mixture thereof, the reaction product comprises HFO-1234yf.

### DETAILED DESCRIPTION OF THE INVENTION

The hydrogenation reactions of the present invention (Hydrogenation Process and Intermediates Process) are preferably carried out in the gas phase in a corrosion-resistance reactor packed with a catalytically effective amount of the catalyst at temperatures and pressures and contact times effective to produce the reaction results desired for the particular reaction. The hydrogenation reactions are preferably carried out at atmospheric pressure or at higher or lower pressures.

The hydrogenation catalyst in each hydrogenation reaction in the Hydrogenation Process consists essentially or Cu, Ru, Cu—Pd, Ni—Cu, or Ni—Pd, with or without a support (carrier). These same catalysts are preferably used in the hydrogenation reactions in the Intermediates Process, although the catalyst used in a particular hydrogenation reaction in the Intermediates Process can be different from the catalyst used in a hydrogenation reaction in the Hydrogenation Process. The catalyst can include a support in each of the hydrogenation reactions. When a support is used as part of the catalyst, the Cu, Ru, Cu—Pd, Ni—Cu, or Ni—Pd can be loaded onto the support in a conventional manner used for loading metals onto supports, including combinations of metals. For example, the catalysts can be prepared by either precipitation or impregnation methods of the Cu, Ru, Cu—Pd, Ni—Cu, or Ni—Pd on a support as generally described in Satterfield on pp. 87-112 in Heterogeneous Catalysts in Industrial Practice, 2^{nd} ed. (McGraw-Hill, New York, 1991). The support is preferably inert, if not positively participative in the obtaining the desired result of the reaction, under the conditions of the reaction. The preferred support is carbon, which may be treated to enhance its support function for the catalyst loaded onto the carbon. One example of treatment is acid washing of the carbon.

When the catalyst is Cu—Pd or Ni—Pd, the Pd is preferably present in a minor amount as compared to the weight of the Cu or Ni. For, example, when the catalyst is Cu—Pd, the loading on the support in one embodiment is 0.1 to 20 wt % Cu and 0.1-1.0 wt % Pd. These same proportions can apply to the Ni—Pd catalyst. When the catalyst if Ni—Cu, the mole ratio of these metals can range from 1:99 to 99:1. In one embodiment, the molar ratio of these metals is about 1:1.

The foregoing description of catalysts applies to each of the hydrogenation reactions encompassed by the Hydrogenation Process and each of the hydrogenation reactions of the Intermediates Process, when these catalysts are used in the Intermediate Process.

Each of the reactant compounds HCFC-235cb, HCFC-225ca, HCFO-1224yd, and CFO-1214ya are commercially available or can be prepared by known processes in varying degrees of purity. Some impurities may participate in the hydrogenation reaction to form intermediates that can be converted directly or indirectly to the desired HFP propene. Other impurities may be unaffected by the reaction.

When the compound is CF₃CF₂CHXCl, wherein X is Cl, i.e. HCFC-225ca, the reaction product of the hydrogenation reaction in the presence of catalyst described above comprises the compound CF₃CF═CH₂ (HFO-1234yf).

This reaction product may also comprise at least one of the compounds CF₃CF═CHCl (HCFO-1224yd) and CF₃CF₂CH₂Cl (HCFC-235cb).

When the compound CF₃CF═CHCl is in the reaction product, this compound can be converted to the compound CF₃CF═CH₂ by hydrogenation in the presence of a catalyst such as described above. The catalyst can be the same or different from the catalyst used to form the reaction product. The hydrogenation of the compound CF₃CF═CHCl to a reaction product comprising CF₃CF═CH₂ is in effect practice of the present invention when the reactant compound is CF₃CF═CXCl, wherein X is H.

When the compound CF₃CF₂CH₂Cl is in the reaction product, this compound can be converted to the compound CF₃CF═CH₂, first by dehydrofluorination of said compound CF₃CF₂CH₂Cl to form the compound CF₃CF═CHCl and then hydrogenation of said compound CF₃CF═CHCl in the presence of catalyst as described above. Alternatively, the compound CF₃CF₂CH₂Cl can be converted to CF₃CF═CH₂ by hydrogenation in the presence of catalyst as described above.

When the reactant compound comprises HCFC-225ca, this compound will typically be accompanied by the compound CF₃CCl₂CHF₂ (HCFC-225aa) in said contacting and reacting in the presence of the catalyst, whereby the reaction product will also comprise at least one of the compounds CF₃CCl═CHF (HCFO-1224xe), CF₃CHClCHF2 (HCFC-235da), CF₃CH₂CF₂H (HCFC-245fa), and CF₃CH═CHF (HFO-1234ze) as reaction products of the hydrogenation of the HCFC-225aa during the hydrogenation of the HCFC-225ca. The HCFO-1234xe and HFC-245fa are both intermediates for the formation of CF₃CH═CHF (HFO-1234ze). The presence of HFO-1234ze in the reaction product means this compound is formed directly from the hydrogenation of HCFC-225aa.

When the reaction product compound is CF₃CCl═CHF (HCFO-1234xe), this compound is hydrogenated in the presence of catalyst as described above to form a reaction product comprising at least one of the compounds CF₃CH═CHF (HFO-1234ze) and CF₃CHClCH₂F (HCFC-234db).

When the reaction product comprises the compound CF₃CHClCH₂F (HCFC-234db), this compound is converted to the reaction product comprising the compound CF₃CH═CHF by dehydrochlorination of the compound CF₃CHClCH₂F.

When the reaction product comprises the compound CF₃CHClCHF₂ (HCFC-235da) this compound is converted to reaction product comprising the compounds CF₃CH═CF₂ or CF₃CH═CHF. In one embodiment, the HCFC-235da can be dehydrochlorinated to form reaction product comprising HFO-1225zc (CF₃CH═CF₂). In another embodiment, the HCFC-235da is reacted with H₂ to remove both Cl and F (hydrodehalogenated) to form reaction product comprising HFO-1234ze.

When the reaction product comprises the compound CF₃CH₂CF₂H (HCFC-245a), this compound can be dehydrofluorinated to form the reaction product comprising CF₃CH═CHF (HFO-1234ze).

When the reactant compound is CF₃CF₂CHXCl, wherein X is H, i.e. (HCFC-235cb), the reaction product with H₂ comprises the compound CF₃CF═CH₂ (HFO-1234yf).

When the reactant compound is CF₃CF═CXCl, wherein X is Cl or CF₃CF═CXCl, wherein X is H, or a mixture of these reactant compounds, the reaction product of the hydrogenation reaction in the presence of catalyst as described above comprises the compound CF₃CF═CH₂. Thus, CFO-1214ya or HCFO1224yd can be the reactant compounds, one without the other, or these compounds can be present as a mixture of reactant compounds.

As used herein, the terms “comprises,” “comprising,” “includes,” “including,” “has,” “having” or any other variation thereof, are intended to cover a non-exclusive inclusion. For example, a composition, process, method, article, or apparatus that comprises a list of elements is not necessarily limited to only those elements but may include other elements not expressly listed or inherent to such composition, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

The transitional phrase “consisting of” excludes any element, step, or ingredient not specified. If in the claim such would close the claim to the inclusion of materials other than those recited except for impurities ordinarily associated therewith. When the phrase “consists of” appears in a clause of the body of a claim, rather than immediately following the preamble, it limits only the element set forth in that clause; other elements are not excluded from the claim as a whole.

The transitional phrase “consisting essentially of” is used to define a composition, method that includes materials, steps, features, components, or elements, in addition to those literally disclosed provided that these additional included materials, steps, features, components, or elements do not materially affect the basic and novel characteristic(s) of the claimed invention, especially the mode of action to achieve the desired result of any of the processes of the present invention.

The term ‘consisting essentially of’ occupies a middle ground between “comprising” and ‘consisting of’.

Where applicants have defined an invention or a portion thereof with an open-ended term such as “comprising,” it should be readily understood that (unless otherwise stated) the description should be interpreted to also include such an invention using the terms “consisting essentially of” or “consisting of.”

Also, use of “a” or “an” are employed to describe elements and components described herein. This is done merely for convenience and to give a general sense of the scope of the invention. This description should be read to include one or at least one and the singular also includes the plural unless it is obvious that it is meant otherwise.

### EXAMPLES

The concepts described herein will be further described in the following Examples, which do not limit the scope of the invention as described in the claims.

### Example 1—Hydrogenation of 225ca to 1234y, 1224yd, and 235cb

10 cc 10% Cu/C is loaded into a ½-inch (1.3 cm) Hastelloy® C 227 reactor. The catalyst is reduced at 250° C. with H₂ for 4 hours. Then HCFC-225ca (GC analysis of HCFC-225ca reactant mixture in Table 1), is fed at 3.11 sccm with H₂ (10.5 sccm) at 325° C. and 350° C. at atmospheric pressure. The reaction product stream from the reactor is analyzed by GC and GC-MS. The result of the hydrogenation reaction is shown in Table 2. HFO-1234yf, HCFO-1224yd and HCFC-235cb are reaction products made in this reaction.

TABLE 1
GC analysis of starting material 225ca mixture

| Compound | 235cb | 225ca | 225aa | 225cb | Others |
|---|---|---|---|---|---|
| Mol % | 1.44 | 83.25 | 4.14 | 11.14 | 0.03 |

TABLE 2
GC analysis of reaction product

| Temp | Organic | H₂ | mol % of product | | | | | | |
|---|---|---|---|---|---|---|---|---|---|
| ° C. | sccm | sccm | 1234yf | 1224yd | 1224xe | 235cb | 225ca | 225aa | 225cb |
| 325 | 3.11 | 10.5 | 1.51 | 1.82 | 2.07 | 3.58 | 73.87 | 1.35 | 11.52 |
| 350 | 3.11 | 10.5 | 4.71 | 6.77 | 2.48 | 10.66 | 49.0. | ND | 12.66 |

### Example 2-Hydrogenation of HCFC-225ca, Cu—Pd Catalyst

10 cc 0.5% Pd-8.5% % Cu/C is loaded into a ½-inch (1.3 cm) Hastelloy® C 227 reactor. The catalyst is reduced at 400° C. with H₂ for 4 hours. Then a 225ca/cb mixture (GC analysis of HCFC-225ca reactant mixture in Table 1), is fed at 3.11 sccm with H₂ (10.5 sccm) at 125, 140 and 160° C. at atmospheric pressure. The stream from the reactor is analyzed by GC and GC-MS. The result of the test is shown in Table 3. HFO-1234yf, CFO-1224yd and HCFC-235cb are made in this reaction.

TABLE 3
GC analysis of reaction product

| Temp | mol % of product | | | | | | |
|---|---|---|---|---|---|---|---|
| ° C. | 1234yf | 1224yd | 1224xe | 235cb | 225ca | 225aa | 225cb |
| 125 | 0.19 | 14.49 | 3.30 | 16.03 | 51.03 | 0.95 | 12.80 |
| 140 | 0.38 | 29.17 | 3.44 | 34.29 | 17.39 | 0.82 | 12.56 |
| 160 | 0.73 | 36.64 | 3.44 | 41.11 | 1.41 | ND | 12.5 |

This Example shows the conversion of 225ca and 1224yd to 1234yf in that the 1224yd is an intermediate to the formation of 1234yf.

### Example 3-Hydrogenation of HCFC-235cb to HFO-1234yf

10 cc 10% Cu/C is loaded into a ½-inch (1.3 cm) Hastelloy® C 227 reactor. The catalyst is reduced at 250° C. with H₂ for 4 hours. Then the 235cb was fed at 3.5 sccm with H₂ (6 sccm) at 325° C. and 350° C. at atmospheric pressure. The stream from the reactor is analyzed by GC and GC-MS. The result of the test is shown in Table 4. The 235cb is converted to 1234yf at a selectivity of 88 to 90%.

TABLE 4
GC analysis of reaction product

| Temp | mol % of product | | |
|---|---|---|---|
| ° C. | 1234yf | 245cb | 235cb |
| 325 | 14.85 | 1.89 | 82.98 |
| 350 | 26.69 | 2.71 | 69.11 |

### Example 4-Hydrogenation of 225ca to 1234y, 1224yd and 235cb, Ni—Cu Catalyst

10 cc Johnson Matthey CP447 Ni—Cu/C is loaded into a ½-inch (1.3 cm) Hastelloy® C 227 reactor. The catalyst is reduced at 400° C. with H₂ for 4 hours. Then 225ca (GC analysis of mixture in Table 5), is fed at 3.11 sccm with H₂ (11 sccm) at 225, 250, 275 and 300 C at atmospheric pressure. The stream from the reactor is analyzed by GC and GC-MS. The result of the test is shown in Table 6. The 1234yf, 1224yd and 235cb are made in this reaction.

TABLE 5
GC analysis starting 225ca mixture
Mole Percent

| 225ca | 225aa | 225cb | Others |
|---|---|---|---|
| 47.43% | 3.09% | 48.53% | 0.95% |

TABLE 6
GC analysis of reaction product (F22E is CF₃CF₂CH═CHCF₂CF₃)

| | Mole Percent | | | | | | | | | | |
|---|---|---|---|---|---|---|---|---|---|---|---|
| Temp | | | | | 1224 | | 1224 | | | | |
| ° C. | 1234yf | 1234ze | F22E | 1224yd-E | isomer 1 | 235cb | isomer 2 | 225ca | 225aa | 225cb | Others |
| 225 | 2.81% | 0.00% | 0.00% | 0.85% | 0.36% | 0.35% | 0.11% | 40.53% | 2.90% | 50.94% | 1.16% |
| 250 | 4.92% | 0.00% | 0.08% | 1.72% | 0.60% | 0.74% | 0.23% | 35.32% | 2.53% | 50.92% | 2.94% |
| 275 | 8.56% | 0.00% | 0.38% | 3.44% | 1.12% | 1.77% | 0.44% | 25.42% | 1.87% | 52.53% | 4.46% |
| 300 | 11.66% | 0.16% | 1.40% | 5.82% | 1.53% | 3.39% | 0.70% | 13.63% | 1.13% | 54.30% | 6.27% |

### Example 5-Hydrogenation of 1214ya to 1234y and 1224yd, Ni—Cu Catalyst

10 cc Johnson Matthey CP447 Ni—Cu/C is loaded into a ½-inch (1.3 cm) Hastelloy® C 227 reactor. The catalyst is reduced at 400° C. with H₂ for 4 hours. Then 1214ya is fed at 3.11 sccm with H₂ (11 sccm) at 225, 250, 275 and 300° C. at atmosphere pressure. The stream from the reactor is analyzed by GC and GC-MS. 1234yf and 1224yd are made in this reaction as shown in Table 7.

TABLE 7
GC analysis of reaction product

| Temp | Mole Percent | | | |
|---|---|---|---|---|
| ° C. | 1234yf | 1224yd | 1214ya | Others |
| 225 | 2% | 1% | 95% | 2% |
| 250 | 18% | 6% | 70% | 6% |
| 275 | 23% | 18% | 50% | 9% |
| 300 | 45% | 23% | 25% | 7% |

### Example 6-Hydrogenation of 1214ya to 1234yf and 1224yd, Pd—Cu Catalyst

10 cc 0.5% Pd-8.5% % Cu/C is loaded into a ½-inch (1.3 cm) Hastelloy® C 227 reactor. The catalyst is reduced at 400° C. with H₂ for 4 hours. Then 1214ya is fed at 3.11 sccm with H₂ (10.5 sccm) at 125, 140 and 160° C. at atmosphere pressure. The stream from the reactor is analyzed by GC and GC-MS. The 1234yf and 1224yd are made as shown in Table 8.

TABLE 8
GC analysis of reaction product

| Temp | mol % of product | | | |
|---|---|---|---|---|
| ° C. | 1234yf | 1224yd | 1214ya | Others |
| 125 | 0.6% | 20% | 69.4% | 10% |
| 140 | 1.2% | 40% | 38.8% | 20% |
| 160 | 3% | 60% | 12% | 25% |

What is claimed is:

1. A process comprising contacting a combined feed of CF₂ClCF₂CHCl₂ (HCFC-225cb) and CF₃CF₂CHCl₂ (HCFC-225ca) with a catalyst and producing HCFO-1224yd in an amount ranging between about 14 mol % and 36 mol % HCFO-1224yd.

2. The process of claim 1 wherein contacting the combined feed and catalyst is conducted at temperature ranging between about 125° C. and 160° C.

3. The process of claim 1 wherein the catalyst comprises one of Cu, Ru, Cu—Pd, Ni—Cu, or Ni—Pd supported on a carrier.

4. A process comprising:
a. activating one of Cu, Ru, Cu—Pd, Ni—Cu, or Ni—Pd supported on a carrier comprising carbon to form an activated catalyst, and
b. contacting a combined feed of CF₂ClCF₂CHCl₂ (HCFC-225cb) and CF₃CF₂CHCl₂ (HCFC-225ca) with the activated catalyst.

5. The process of claim 4 wherein activating the catalyst comprises reducing the catalyst in the presence of hydrogen at temperatures ranging from 250° C. to 400° C.

6. A process comprising contacting a combined feed of CF₂ClCF₂CHCl₂ (HCFC-225cb), CF₃CF₂CHCl₂ (HCFC-225ca) and CF₃CCl₂CHF₂ (HCFC-225aa) and a catalyst and producing a product mixture CF₃CF₂CH═CHCF₂CF₃ (HFO-F22E).

7. The process of claim 6 wherein the mixture further includes (i) at least one of HCFO-1224yd(E), HCFO-1224yd(Z), and HCFC-235da and (ii) unreacted HCFC-225cb, HCFC-225ca and HCFC-225aa.

8. The process of claim 7 wherein the contacting is conducted at a temperature range of 225° C. to 300° C.

9. The process of claim 6 wherein further comprising:
a. first activating one of Cu, Ru, Cu—Pd, Ni—Cu, or Ni—Pd supported on a carrier to form an activated catalyst, and
b. contacting the combined feed of CF₂ClCF₂CHCl₂ (HCFC-225cb) and CF₃CF₂CHCl₂ (HCFC-225ca) with the activated catalyst.

10. The process of claim 9 wherein activating the catalyst comprises reducing the catalyst in the presence of hydrogen at temperatures ranging from 250° C. to 400° C.

11. A composition comprising HCFO-1224yd and at least one of HCFC-225ca and HCFC-225cb, and the amount of HCFO-1224yf is greater than the total amount of HCFC-225ca and HCFC-225cb.

12. A composition comprising CF₃CF₂CH═CHCF₂CF₃ (HFO-F22E), (i) at least one of HCFO-1224yd(E), HCFO-1224yd(Z), and HCFC-235da, and (ii) at least one of HCFC-225cb, HCFC-225ca and HCFC-225aa.

13. The process of claim 4 further comprising loading one of the Cu, Ru, Cu—Pd, Ni—Cu, or Ni—Pd supported on a carrier in a reaction zone prior to activation.

14. The process of claim 13 wherein the reaction zone comprises a corrosion-resistance reactor.

15. The process of claim 3 wherein the carrier comprises carbon.
